(19) United States
(12) Patent Application Publication
(10) Pub. No.: US 2023/0364311 A1
(43) Pub. Date: Nov. 16, 2023

(54) APPARATUS AND METHODS FOR ASSISTING BREASTFEEDING

(71) Applicant: Omid Options LLC, Great Falls, VA (US)

(72) Inventor: Amin AKHLAGHI, Great Falls, VA (US)

(73) Assignee: Omid Options LLC, Great Falls, VA (US)

(21) Appl. No.: 18/312,836

(22) Filed: May 5, 2023

Related U.S. Application Data
(60) Provisional application No. 63/364,536, filed on May 11, 2022.

(51) Int. Cl. A61M 1/06; A61M 39/10

(52) U.S. Cl. CPC A61M 1/067; A61M 1/064; A61M 39/10

(57) ABSTRACT

Apparatus and methods for assisting breastfeeding are disclosed where the device is a feeding aid which attaches to an individual in a hands-free manner so that an infant can latch on and suck either expressed breastmilk or formula. The device provides a raised surface that allows the baby to latch and which contains openings which are connected to a tube that originates at a reservoir that holds the desired liquid such as expressed breastmilk or formula for the nourishment of the baby. Several openings can serve as a conduit for the suction to allow for the sensation of breastfeeding and stimulation of the mother's nipple and areola to promote breast milk production. A flow regulator may adjust the flow of the liquid coming to the baby at each feeding.

### CROSS-REFERENCE TO RELATED APPLICATIONS

This application claims the benefit of priority to U.S. Prov. App. 63/364,536 filed May 11, 2022, which is incorporated herein by reference in its entirety.

### FIELD OF THE INVENTION

The present invention relates to medical devices. In particular, the present invention relates to apparatus and methods for supporting breastfeeding parents by allowing a baby to consume formula or breastmilk while latching on to the breast of the breastfeeding parent via a nipple shield with an incorporated feeding device.

### BACKGROUND OF THE INVENTION

Struggles with breastfeeding have long been a taboo subject and remain unspoken while at the same the pressure on women to breastfeed continues to increase. The benefits of breastmilk as well as the act of breastfeeding for mother and child are not in dispute. Breastfeeding helps mothers bond with the baby and allows the baby to take in important nutrients that have established health benefits. Breastfeeding also provides the benefit of bonding by enabling skin-to-skin contact.

Breastfeeding is particularly problematic for women who are either unable to produce enough or any milk or who are unable to properly latch the baby to their breast. This is also problematic for parents such as adoptive parents, women who have undergone mastectomies, same sex couples, or any other parental figures who desire to play a role in early bonding and feeding such as fathers.

Conventional devices which are designed to facilitate breastfeeding or to supplement milk intake by the infant during breastfeeding are inefficient and can require the help of at least one additional person such that use of these devices becomes impractical and inconvenient. Examples of some conventional products can include breast pumps, nipple shields, etc. Breast pumps assume that the mother has the ability to produce breast milk; however, these devices express breast milk into a bottle for later feeding forcing the mother to forego the skin-to-skin bonding experience with the baby. Nipple shields aide with babies who are unable to latch but also assume that the mother has the ability to produce enough breast milk to adequately nourish the child.

Other breastfeeding aide devices are impractical as they generally require the breastfeeding parent to insert a feeding tube into the baby's mouth while the baby either latches on through a nipple shield or simply sucks from the tube. However, the feeding tube typically has a tendency to fall out of the baby's mouth as it is not secured in any manner and any movement or tugging upon the tube tends to dislocate the feeding tube.

Accordingly, there is a need for a breastfeeding device which can either directly feed the baby during breastfeeding or supplement breastfeeding with the baby latched while allowing for direct skin-to-skin contact between the baby and the breastfeeding parent.

### SUMMARY OF THE INVENTION

The devices and methods described combine as many benefits of breastfeeding as possible and also aides the mother in increasing her milk production while feeding formula or another fluid nutrient to the baby as a partial supplement to their own breast milk or even as a substitute while breastfeeding. Mothers may choose to feed expressed breastmilk if the baby does not latch and parents who cannot produce enough or any breast milk can feed the baby formula while still benefiting from the skin-to-skin bonding.

In one variation, the device can include a nipple shield which may attach to the breast or chest of the breastfeeding individual via a tacky substrate or an adhesive such as a reusable adhesive. A thin feeding tube through which the baby may receive either partial or complete feeding with either expressed breast milk and/or formula (or other fluid nutrient) may be incorporated within or upon the nipple shield and fluidly connect to a reservoir which holds the milk or formula and is designed to function in a handsfree manner. The reservoir may include a valve such as a unidirectional valve (e.g., duck-bill valve) that allows the reservoir to drain via the tube while preventing backflow or spillage and further removes any need to manually push or urge the milk or formula into the tube for baby to feed.

The nipple shield through which the feeding tube is incorporated may be configured to allow for the greatest areola exposure so that maximum areola stimulation from the sensation of suction by the baby can be achieved. The nipple shield may allow for the sensation of suction to also be felt by the nipple of the individual wearing the nipple shield and thereby promote milk production as provided by the suction.

One variation of the breastfeeding aid device may generally comprise a reservoir having a first opening, a flow selector positioned within the first opening, wherein the flow selector is configured to provide a predetermined flow rate from the reservoir, and a nipple shield configured to be positioned over a nipple of a subject such that the nipple is sealingly contained within a cavity defined by the nipple shield, the cavity having a channel fluidly coupled to the distal opening of the reservoir via the at least one fluid lumen and the cavity having at least one additional channel defined through a wall of the cavity. A suction force applied upon an exterior of the cavity may draw a fluid from within the reservoir and through the channel via the at least one fluid lumen and simultaneously creates a negative pressure within the cavity to stimulate the nipple of the subject.

One variation for a method of aiding breastfeeding may generally comprise securing a nipple shield over a nipple of a subject such that the nipple is sealingly contained within a cavity defined by the nipple shield, receiving a suction force imparted upon an exterior of the cavity such that a fluid contained within a reservoir is drawn via a negative pressure through at least one fluid lumen fluidly coupled to the reservoir and through a channel defined within a wall of the cavity while the fluid is metered via a unidirectional valve positioned within the reservoir, and further receiving the suction force within the cavity such that the nipple of the subject is simultaneously stimulated.

### BRIEF DESCRIPTION OF THE DRAWINGS

FIG. 1 shows a perspective assembly view of one assembled variation of the device.

FIGS. 2A and 2B show perspective views of one variation of the nipple shield assembly which is connectable to the reservoir assembly.

FIGS. 3A to 3C show front and partial cross-sectional side views of the crown or cavity of the nipple shield and integrated fluid lumen.

FIG. 3D shows a partial cross-sectional side view of another variation including an insert for blocking the openings to the nipple.

FIG. 3E shows a partial cross-sectional side view of another variation where the lumen within the crown may vary its cross-sectional area.

FIGS. 4A and 4B show front views of the crown or cavity having different variations of the openings.

FIGS. 5A to 5C show front views of the crown or cavity illustrating the directional collapsibility.

FIG. 6 shows an example of how the fluid lumen can be configured to collapse during use when the reservoir assembly is fluidly coupled to the nipple shield assembly.

FIG. 7 shows an example of a nipple shield (contact-type) having an adhesive sealing layer.

FIGS. 8A and 8B show perspective views of a variation of the nipple shield detached from the adhesive sealing layer.

FIGS. 9A and 9B show perspective views of the reservoir assembly having an adjustable clamping mechanism positioned along the fluid lumen for metering a flow of fluid through the fluid lumen.

FIGS. 10A to 10C show perspective and side views of one variation of the reservoir having a valve removably positioned within or along the reservoir.

FIGS. 11A to 11C show perspective, exploded assembly, and detail perspective views of an alternative variation of a flow regulation mechanism.

FIG. 12 shows one variation of how the device is positionable relative to a mother breastfeeding an infant.

### DETAILED DESCRIPTION OF THE INVENTION

The breastfeeding device allows individuals who may be challenged in breastfeeding or otherwise unable to breastfeed to experience the benefits of breastfeeding by enabling skin-to-skin bonding with the breastfeeding infant and encouraging the release of oxytocin while providing necessary nourishment to the infant. The device further enables the individual to feed the infant while handsfree and also allows for adjustment of the fluid flow rate of the milk or fluid by a flow limiting mechanism as well as by the action provided by the infant as well. Additionally, the nipple shield assembly may be adhered to the skin of the breastfeeding individual and may also provide for a differential nipple wall thickness which may enable the adjustable collapse of the crown or cavity of the nipple shield, as described in further detail.

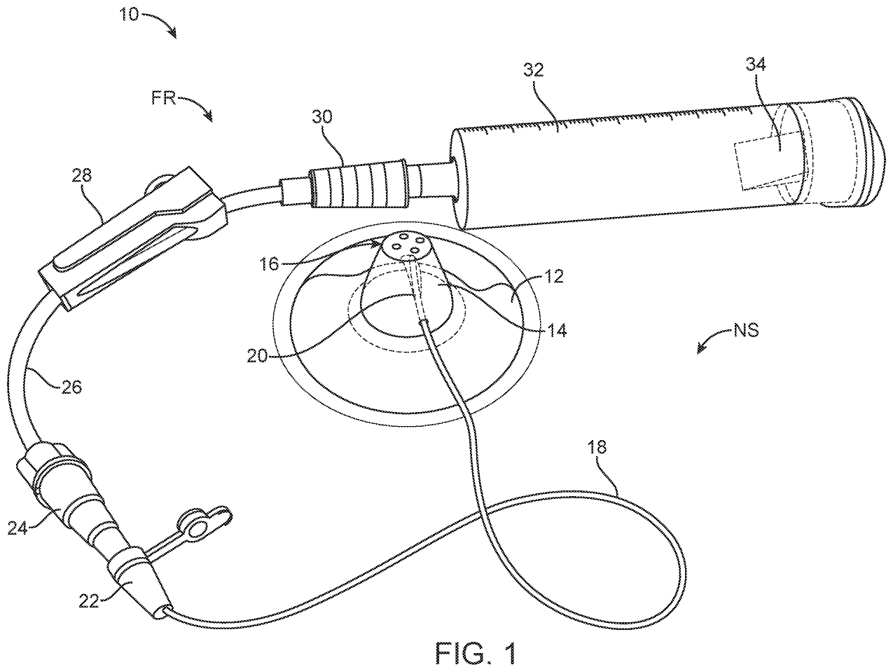

FIG. 1 illustrates a perspective assembly view of one assembled variation of the device 10 where a nipple shield assembly NS may be fluidly coupled to a fluid reservoir assembly FR. One variation of the nipple shield 12 is shown having a crown or cavity 14 formed into a raised portion for encapsulating the nipple of the breastfeeding individual. The raised surface may have one or more openings 16 which are defined through the wall of the distal portion of the crown or cavity 14 for communication with the interior of the crown or cavity 14. At least one opening may be fluidly coupled to a fluid lumen 18 which may be attached, integrated, or otherwise formed within a side wall of the crown or cavity 14 for fluid coupling to the opening, as shown. The fluid lumen 18 may have a flexible elongate length with a proximal end having a first coupling or connector 22 which may be removably attached to a second coupling or connector 24, such as a luer fitting or coupling, of the fluid reservoir assembly FR. In other variations, the first coupling or connector 22 may be omitted entirely and the proximal end of the fluid lumen 18 may instead be simply friction-fitted over the second coupling or connector 24. The second coupling or connector 24 may have second fluid lumen 26 which is removably coupled via a third coupling or connector 30 to a fluid reservoir 32 which may hold or otherwise retain a volume of fluid within such as milk and/or fluid nutritional supplement for feeding the infant. The fluid reservoir 32 may be comprised of any fluid container such as a syringe body, flexible pouch, etc. and the reservoir 32 may further define a plurality of gradations for indicating the fluid volume within.

In yet other variations, the couplings 22, 24 may be omitted and the fluid lumen 18 may form a continuous length for coupling directly to the fluid reservoir 32 either with the third coupling or connector 30 or without the coupling or connector 30 by directly integrating the fluid lumen 18 with the fluid reservoir 32.

The nipple shield assembly NS may be readily removed from the fluid reservoir assembly FR by disengaging the couplings 22, 24 from one another to enable the removal of either the fluid reservoir assembly FR and/or nipple shield assembly NS from the individual while other assembly may remain upon or secured to the individual.

The flow of the fluid dispensed from the fluid reservoir 32 may be actuated or initiated by the infant sucking upon the crown or cavity 14 of the nipple shield 12 such that a negative pressure is created within the interior of the crown or cavity 14. This negative pressure is formed throughout the length of the fluid lumen 18, 26 to draw the fluid within the fluid reservoir 32. To regulate the fluid flow, an adjustable clamping mechanism 28 such as a screw clamp, pinch clamp, roller clamp, etc. may be placed upon a portion of the second fluid lumen 26 to adjust or meter the flow of fluid exiting the fluid reservoir and passing through the second fluid lumen 26. Additionally, a valve 34 such as a unidirectional valve may be positioned within or along the fluid reservoir 32 to further meter the flow of fluid from the reservoir 32. As the infant sucks upon the crown or cavity 14, the negative pressure may draw the fluid from the reservoir 32 and the valve 34 may allow for the ambient air to pass through the valve 34 and into the reservoir 32 each time the infant sucks upon the nipple shield. Every time the infant ceases sucking, the valve 34 may remain in a closed configuration while maintaining a constant pressure within the reservoir 32 to prevent any further fluid from flowing out of the reservoir inadvertently thereby metering the fluid into boluses mimicking the natural flow of milk from the mother's breast.

The clamping mechanism 28 may also be adjusted to further meter the flow volume or flow rate of the fluid from the reservoir 32 during infant feeding. As the primary driver of the fluid release from the reservoir 32 is the degree of suction provided by the feeding infant, the clamping mechanism 28, valve 34, and/or collapsibility of the first fluid lumen 18, as described in further detail here, may provide for adjustability of the flow rate of the fluid from the reservoir 32.

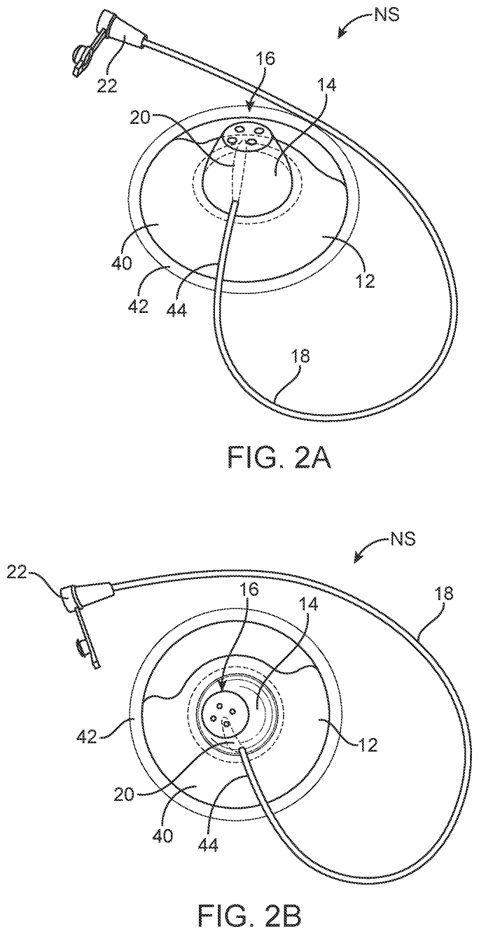

FIGS. 2A and 2B show various perspective views of one variation of the nipple shield assembly NS which is connectable to the fluid reservoir assembly FR. As shown, the first coupling or connector 22 may be detached from the fluid reservoir assembly FR allow for the nipple shield assembly NS to remain upon the individual while breastfeeding and allows for the fluid reservoir assembly FR to be removed or adjusted independently of the nipple shield assembly NS. The nipple shield itself 12 may include a flange or brim 42 which may extend circumferentially entirely around the crown or cavity 14 or partially around the crown or cavity 14.

The fluid lumen 18 may be attached, secured, or otherwise integrated along a portion 20 of the flange or brim 42 such that the terminal end of the fluid lumen 18 is fluidly coupled to at least one of the openings 16 along the crown or cavity 14 such that fluid from the fluid reservoir 32 may pass through the fluid lumen 18 and through the connected opening.

A separate adhesive layer 40 may be configured to adhere between a surface of the flange or brim 42 and against the skin of the individual such that the adhesive layer 40 encircles entirely around or partially around the nipple and seals the nipple shield 12 to the breast of the individual such that the crown or cavity 14 contains the nipple of the individual. The adhesive layer 40 may accordingly be configured into a shape which follows the flange or brim 42 of the nipple shield 12 while remaining open in a center portion to allow for insertion of the nipple into the crown or cavity 14. Hence, the adhesive layer 40 may take on any number of shapes such as a circular ring, ovular, rectangular, square, etc. Additionally, the adhesive layer 40 may be fabricated from any number of biologically compatible adhesives or tacky materials such as silicone so long as the layer 40 enables the nipple shield 12 to remain secured against the surface of the skin during breastfeeding and removed afterwards. Another variation may include an adhesive layer 40 which may be formed of a waxy or tacky substance, e.g., lanolin, which may be applied directly upon the breast prior to placing the nipple shield 12 upon the breast to form a temporary adhesive interface. Furthermore, the adhesive layer 40 may be removably attached to the nipple shield 12 for replacement or sterilization or the adhesive layer 40 may be formed as a permanent adhesive attached to the nipple shield 12.

In other variations, the fluid lumen 18 may instead be coupled directly to the flange or brim 42 of the nipple shield 12 itself and the flange or brim 42 may integrate or define a channel or fluid lumen through the flange or brim 42 to at least one of the openings 16 along the crown or cavity 14. Because the fluid lumen 18 is separate from the channel or fluid lumen integrated through the flange or brim 42, the fluid lumen 18 may be optionally decoupled from the flange or brim 42, e.g., for exchanging components or cleaning, etc.

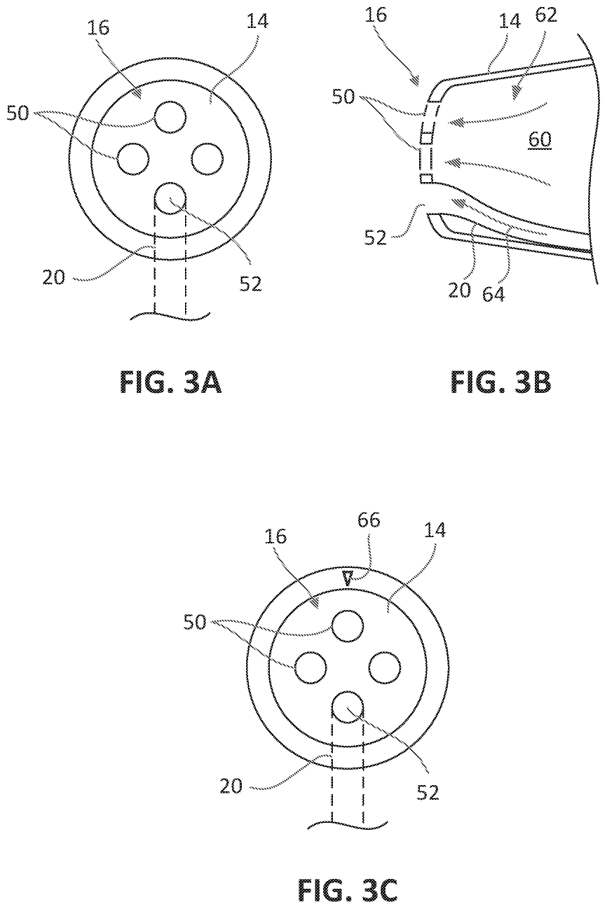

The crown or cavity 14 may define one or more openings 16 along the raised surface of the nipple shield 12, as shown in the detailed top and partial cross-sectional side views of FIGS. 3A and 3B. FIG. 3A illustrates one or more openings 50 which are defined through the wall of the crown or cavity 14 to allow for fluid communication from the cavity interior 60 with the exterior of crown or cavity 14. With the nipple of the breastfeeding individual positioned within the cavity 60, the suction 60 provided by the infant once latched onto the crown 14 may allow for any milk produced by the individual to be drawn into the cavity 60 and out through the one or more openings 50 to feed the breastfeeding infant, as shown in FIG. 3B.

An additional supplemental opening 52 may also be defined along the crown or cavity 14 but supplemental opening 52 may be fluidly coupled via fluid lumen 18 to the fluid reservoir 32. FIG. 3B illustrates how the fluid lumen 18 may be attached or otherwise integrated 20 along the interior of the crown or cavity 14 such that the terminal opening is fluidly coupled to the supplemental opening 52. The fluid lumen is positioned so as to prevent any interference to the infant during breastfeeding. As the infant sucks, the fluid 64 within the fluid reservoir 32 may be drawn through the fluid lumen 18 and out of the supplemental opening 52 to feed the infant. With the openings 50, 52 adjacent to one another, the infant may suck upon the crown 14 so that the negative pressure formed may simultaneously draw the supplemental fluid 64 through supplemental opening 52 while also drawing any milk produced by the breast from the nipple retained within the crown and cavity 14 and through the openings 50 for feeding the infant. The suction 62 formed within the interior cavity 60 may also accordingly stimulate the nipple naturally to produce breastmilk so that the infant may receive the milk directly from the nipple through the natural stimulation provided. In the event that the breast is producing an insufficient supply of milk or no milk at all, the fluid 64 drawn through the supplemental opening 52 may still provide nutrition to the infant while also providing the benefits of breastfeeding to both the infant and breastfeeding individual. Alternatively, in the event that the breast is producing a sufficient supply of milk, the flow of fluid 64 through supplemental opening 52 may be reduced via the adjustable clamping mechanism 28 or ceased entirely while the nipple shield 12 may still be used to reduce any irritation to the nipple.

In yet another variation as shown in FIG. 3D, the openings 50 which extend into the interior cavity 60 for stimulating the nipple may be blocked or obstructed using, for example, an insert I or plug which may be removably inserted within the interior cavity 60. The insert I may obstruct the openings 50 to prevent the suction from the infant from stimulating the nipple and the insert I may also define an interior for accommodating the nipple within. While the insert I is shown as being positioned within the cavity 60, another variation may instead be positioned along an exterior of the crown or cavity 14 to obstruct the openings 50 so long as the covering is prevented from being inserted entirely within the mouth of the infant. Yet another variation of the device may incorporate a crown or cavity 14 which omits the openings 50 entirely so that only the supplement opening 52 is defined along the crown or cavity 14.

Another variation is illustrated in the partial cross-sectional side view of FIG. 3E illustrating how the fluid lumen within the cavity 60 may be varied in its cross-sectional area. In this variation, the lumen may have a first width or cross-sectional area which widens to a second width or cross-sectional area 20′ within the cavity 60. This second width 20′ may then narrow again to the first width or cross-sectional area (or another width smaller than the second width 20′) prior to the supplemental opening 52. In this manner, the lumen shape may replicate the peristaltic pumping of milk through the lumen due to the pressure drop within the second width 20′ and narrowing back to the first width before the fluid exits the supplemental opening 52.

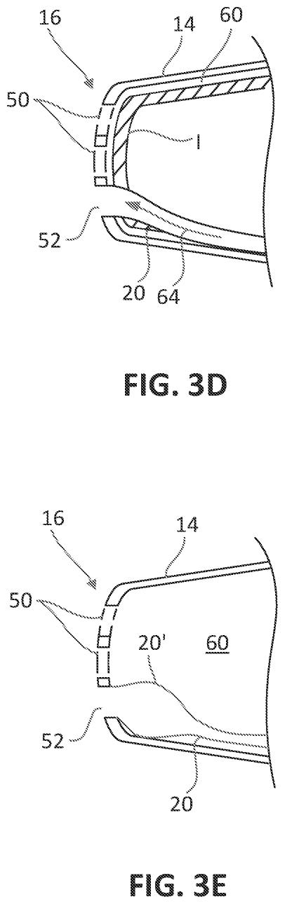

While three openings 50 are shown defined through the interior cavity 60 and a single supplemental opening 52 is shown fluidly coupled to the fluid reservoir 32, these are intended as illustrative examples. Other variations may include any number of openings 50 which are greater than three or fewer than three or these openings 50 may be omitted entirely. Likewise, the single supplemental opening 52 may include more than one opening fluidly coupled to the fluid reservoir in other variations. Furthermore, additional supplemental openings 52 may be fluidly coupled to a single reservoir 32 or additional reservoirs which may hold the same or other supplemental fluids (e.g., medications, electrolytic fluids, etc.) which may be used in any number of combinations when feeding the infant. FIG. 4A illustrates a front view of the crown 14 illustrating a total of three openings 16′ and FIG. 4B illustrates a front view illustrating a total of six openings 16″ as examples of how the various openings may be arranged in a number of different configurations.

The fluid lumen 18, fluid reservoir 32, and other components are positioned away from the face of the infant so as not to interfere with the breastfeeding process. The nipple shield 12 may also be configured for placement upon the breast and nipple in a predetermined orientation depending upon the positioning of the infant during breastfeeding. For instance, the supplemental opening 52 may be positioned relative to the infant so that the supplemental opening 52 is located along the lower side of the infant's mouth when feeding. This may help to ensure that the supplemental opening 52 remains fully patent as the infant latches upon the crown or cavity 14 and deforms the cavity. Hence, the nipple shield 12 may be placed upon the breast and nipple in a particular orientation depending on how the infant is positioned relative to the breast, e.g., lying down, reclined, etc. A orientation indicator 66 such as a line, image, or any marker may be located along the exterior of the crown 14 to indicate where the supplemental opening 52 is located so that the individual may orient the nipple shield 12 accordingly relative to the infant. The example shown in FIG. 3C illustrates the orientation indicator 66 along the exterior of the crown 14 at a location opposite to the supplement opening 52 so that the individual can orient the nipple shield 12 while looking down at the infant's mouth.

Furthermore, the nipple shield 12 can be configured for use on either the left or right breast while in other configurations, one variation of the nipple shield 12 can be configured for the left breast while another variation of the nipple shield 12 can be configured for the right breast.

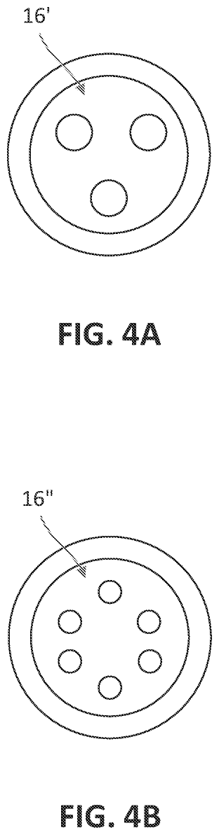

As the infant takes the crown or cavity 14 within their mouth, their natural inclination when latching for breastfeeding is to press down upon the nipple so that the nipple takes on an ovular or compressed configuration to conform to the mouth of the infant. An example is shown between the front views of FIG. 5A, which shows an uncompressed crown 14, and FIG. 5B, which shows a crown 14′ compressed in an ovular configuration when compressed 54 by the mouth of the infant to replicate the natural tendency of the nipple to compress within the infant's mouth.

Each of the openings 16′ are shown to correspondingly compress such that the openings 50′ and supplemental opening 52′ are compressed into ovular configurations. To facilitate the reconfiguration of the crown 14, portions of the wall forming the crown 14 may be configured to have differing wall thicknesses. FIG. 5C shows one variation where the upper and lower portions 56 of the crown 14 may be formed with a first thickness while the side portions 58 of the crown 14 may be formed to have a second thickness which are relatively thinner than the first thickness. When the infant latches upon the upper and lower portions 56, the side portions 58 may accordingly flex preferentially to reconfigure into the oval shape illustrated in FIG. 5B so that the crown 14 collapses its shape into a relatively wider configuration when compressed externally. The presence of the orientation indicator 66 along the exterior of the crown 14 described above may facilitate the positioning of the nipple shield 12 accordingly.

While the crown or cavity 14 may be formed generally into a tapered structure shaped for insertion into the mouth of the infant, the structure may be varied in any number of ways to promote or stimulate primitive neonatal reflexes which may support breastfeeding. For example, the crown 14 may incorporate a cut-out along a portion of the structure. Alternatively, the crown 14 and/or flange or brim 42 of the nipple shield 12 may be contoured in various configurations to promote or stimulate the primitive neonatal reflexes of the infant.

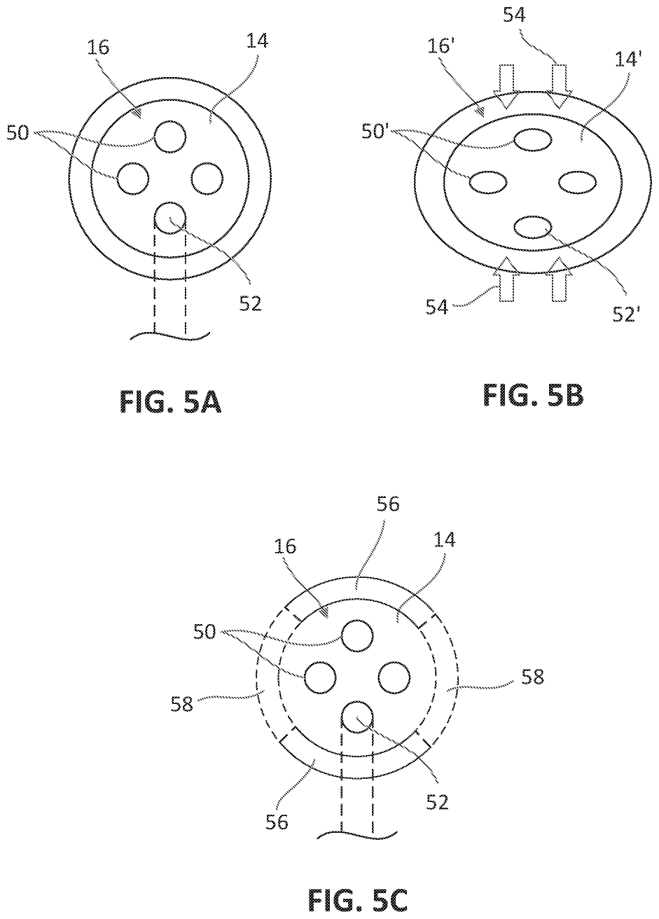

Another optional feature of the device is shown schematically in FIG. 6 to illustrate the selective collapsibility of the fluid lumen 18 during breastfeeding. Each time the infant sucks upon the crown 14, the fluid lumen 18 may be configured to collapse upon application of the negative pressure applied through the supplemental opening 52. The walls of the fluid lumen 18 may be configured along a portion of the length or along the entire length of the fluid lumen 18 to have a flexibility which enables the fluid lumen 18 to collapse. The fluid from the fluid reservoir 32 may be metered into boluses as the suction is applied, a small volume of fluid is allowed to pass through the fluid lumen 18 before the lumen 18 collapses to prevent or restrict the further passage of fluid. When the suction is released, the walls of the fluid lumen 18 may rebound back 68 into its opened configuration to allow for further introduction of additional fluid so that this process may be repeated to replicate the natural process of milk delivery from the breast. This may also help to naturally motivate the infant to continue with breastfeeding.

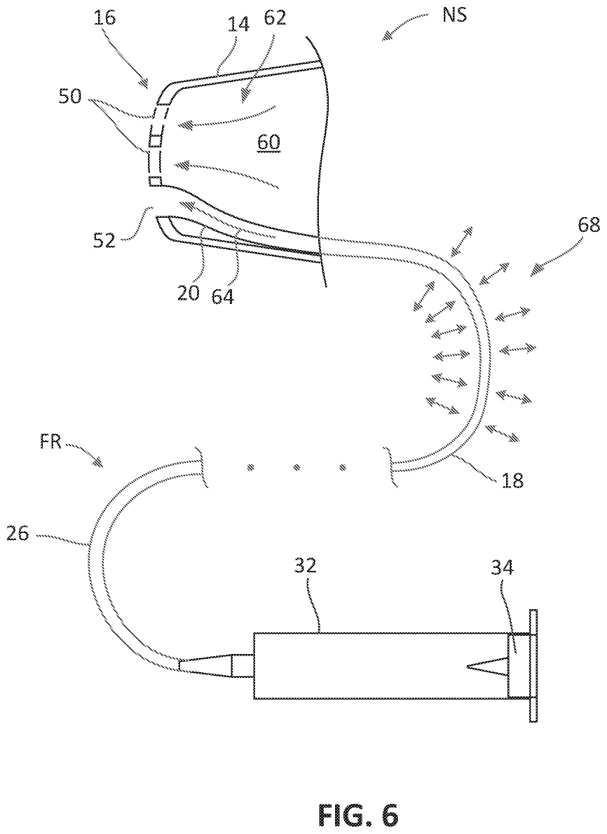

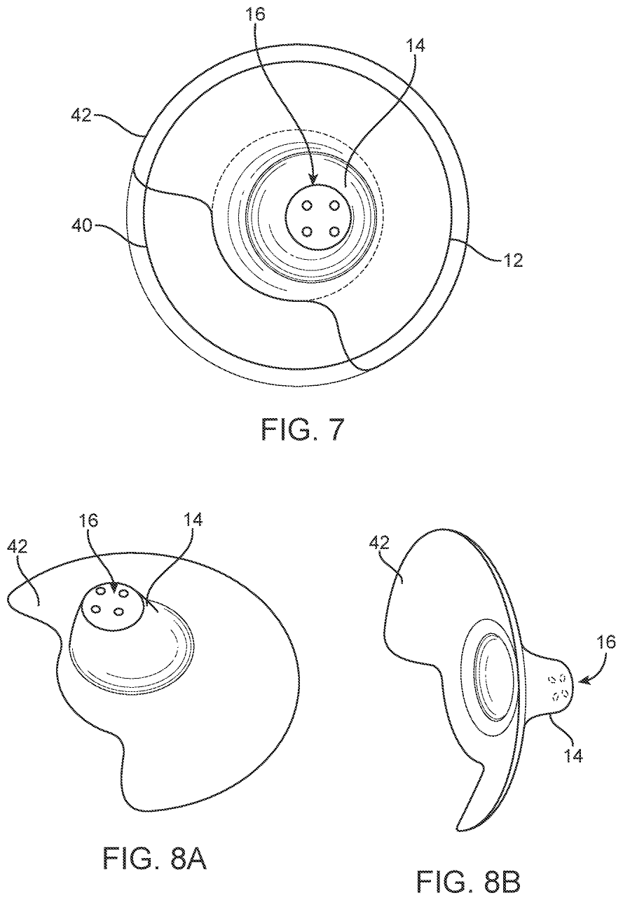

As described above, the flange or brim 42 of the nipple shield 12 may be configured to have a circumferential shape while other variations may have the flange or brim 42 partially removed. FIG. 7 shows a front view of the nipple shield 12 with circumferential adhesive layer 40 attached to illustrate how a partial nipple shield 12 may incorporate the adhesive layer 40. FIGS. 8A and 8B show various perspective views of the nipple shield 12 with the adhesive layer 40 removed to illustrate the configuration of the nipple shield 12, which may be fully transparent or partially transparent over the entire nipple shield 12 or portions of the nipple shield 12.

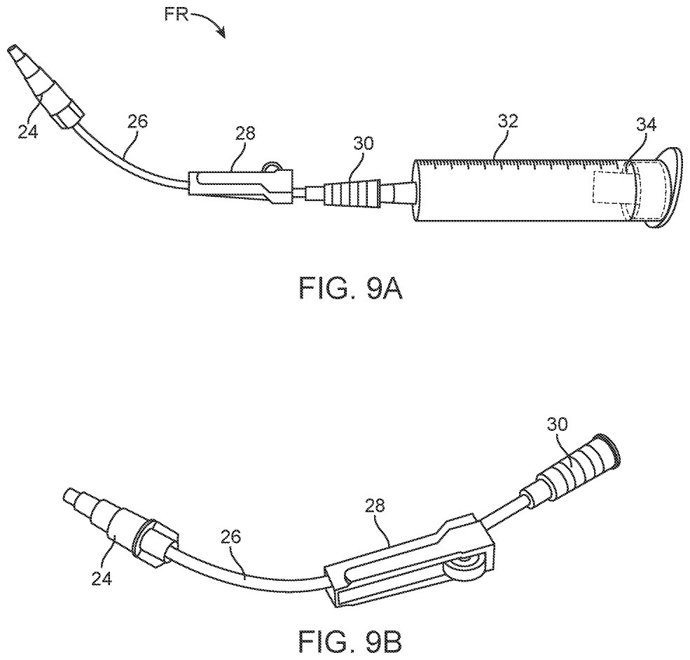

Turning now to the fluid reservoir assembly FR, FIG. 9A shows a perspective assembly view of the assembly with the fluid reservoir 32 and FIG. 9B shows a perspective assembly view with the fluid reservoir 32 removed from the third coupling or connector 30. As shown, the fluid reservoir 32 may be removably connected to the second fluid lumen 26 via the third coupling or connector 30 to allow for the removal of the fluid reservoir 32 during feeding for refilling or replacement. The adjustable clamping mechanism 28 is further shown positioned along the second fluid lumen 26 although the clamping mechanism 28 may be alternatively positioned along the first fluid lumen 18 or integrated with the fluid reservoir 32 instead.

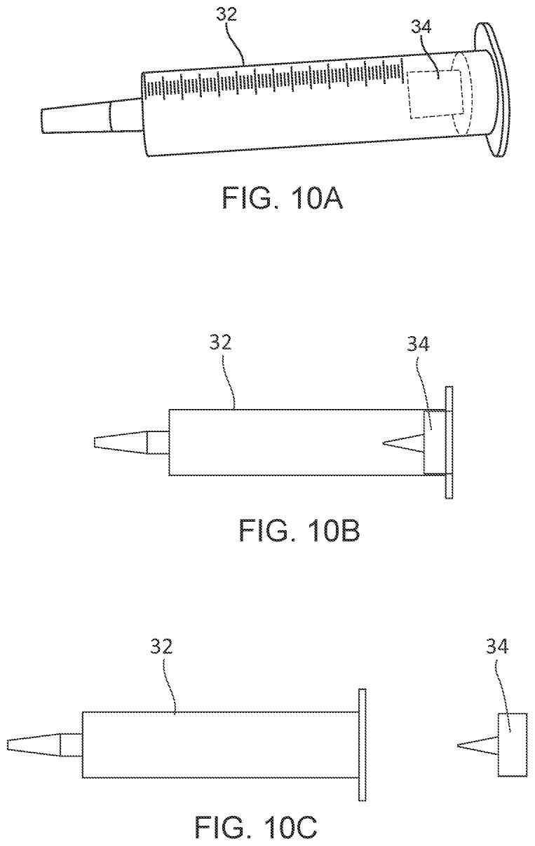

FIGS. 10A to 10C show perspective and side views of the fluid reservoir 32 and valve 34 which may be removably positioned along the reservoir 32 such as the proximal position of the reservoir 32. The fluid reservoir 32 may be configured for placement or securement upon the breastfeeding individual or in proximity but in either case, the fluid reservoir 32 may be detachably coupled so that the individual may feed the infant in a handsfree manner so that the fluid reservoir 32 does not need to be held separately or actuated manually (such as via a plunger) to enable the flow of the fluid form the reservoir. The valve 34 itself is also removable so that the reservoir 32 and valve may be flushed and cleaned for re-use. As previously noted, the fluid reservoir 32 may be comprised of any fluid container such as a syringe body (or any other rigid reservoir), flexible pouch (or any other flexible reservoir), etc. and the reservoir 32 may further define a plurality of gradations for indicating the fluid volume within.

Furthermore, because the fluid reservoir 32 does not need to be manually actuated to allow for the fluid to flow from the reservoir 32, the reservoir 32 may utilize the natural suction provided by the infant and may also take advantage of gravity to create a pressure head within the reservoir 32 by elevating the location of the fluid reservoir 32 relative to the infant. Hence, the fluid reservoir 32 may be positioned or secured to the individual at a location above the infant, such as the shoulder, chest, etc. such that the infant is located below the fluid reservoir 32. The height of the fluid reservoir 32 may vary or may be varied depending upon the infant and individual positioning during breastfeeding.

In another alternative variation, the fluid reservoir 32 may optionally incorporate a mechanism for increasing a pressure within the reservoir 32 to facilitate emptying of the reservoir 32 for infants who may need assistance with suction (e.g., premature infants). Mechanisms such as a plunger or pump may be used which can be actuated automatically or manually to pump air into the fluid reservoir 32 to increase the internal pressure. A pressure indicator may be optionally incorporated to monitor the pressure increase within the reservoir 32.

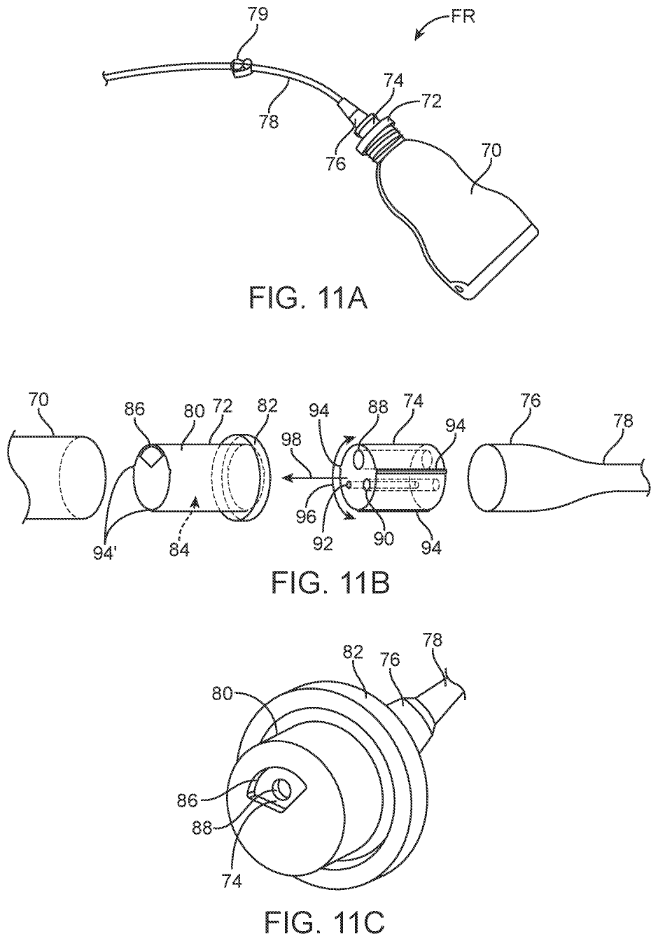

In yet another alternative variation, the fluid reservoir assembly FR may incorporate an alternative flow regulation mechanism, as shown in the perspective view of FIG. 11A. The variation shown illustrates a fluid reservoir 70 which is illustrated as a soft-walled, expandable reservoir or bottle but may be configured as any of fluid reservoirs described herein. The fluid reservoir 70 includes an opening through which a selector coupling or connector 72 may be secured to maintain a fluid tight seal. A flow selector 74 mechanism which defines one or more lumens each having a different diameter through the length of the flow selector 74 may be rotatably retained within the selector coupling or connector 72. A distal end portion of the flow selector 74 may project from the selector coupling or connector 72 where a coupling or connector 76 may form a detachable fluid seal with the flow selector 74 and extend in a fluid lumen 78 for fluidly coupling to the nipple shield assembly NS. An additional lumen clamp 79 may be positioned upon an exterior of the fluid lumen 78 to stop or reduce flow from the reservoir 70 and through the lumen 78. Alternatively, an adjustable clamping mechanism 28 may be placed along an exterior of the lumen 78 to provide further adjustments in the flow rate through the lumen 78.

FIG. 11B shows an exploded assembly view of the flow reservoir assembly FR. The flow selector 74 may be generally configured into a cylindrical body through which one or more openings each with a different diameter may extend through the length of the selector 74. The example shown illustrates a first opening 88 having an opening with a first diameter extending through the entire length of the selector 74. A second opening 90 having an opening with a second diameter which is less than the first diameter may extend in parallel through the length of the selector 74 and a third opening 92 having an opening with a third diameter which is less than the second diameter may extend in parallel through the length of the selector 74. Each of the openings 88, 90, 92 may be uniformly positioned radially apart from one another with each opening being sequentially larger in diameter. The selector body may further define one or more projections 94 which may extend along the length of the body of the selector 74 or extend at least partially where each of the projections 94 may be equally spaced apart from one another over a circumference of the selector 74. While the flow selector 74 shows three openings, any number of openings of different diameters may be included such as flow selectors 74 having fewer than three openings or more than four openings where each opening has a different diameter from one another. Furthermore, three projections 94 are shown extending along the length of the flow selector 74; however, other variations may include fewer than three projections or more than four projections.

The selector coupling or connector 72 may include a receiver body 80 which defines a receiving channel 84 into which the flow selector 74 may be inserted in a snug or fluid tight coupling. As the selector coupling or connector 72 may be fabricated from a soft material such as silicone, rubber, etc., the flow selector 74 may be slidingly inserted into the selector coupling or connector 72 such that the interior surface of the receiving channel 84 may conform against the outer surface of the flow selector 74 in a fluid tight seal. The interior of the receiving channel 84 may further define receiving grooves or channels 94′ which may receive each of the projections 94 along the exterior of the flow selector 74 in a corresponding manner. A distal surface of the receiver body 80 may further define a flow opening 86 selectively located over the distal surface such that when the flow selector 74 is inserted into the receiving channel 84, the projections 94 may urge or automatically align one of the openings of the flow selector 74 to the flow opening 86, as further shown in the perspective view of FIG. 11C.

During use, the flow selector 74 may be inserted, as shown by the directional arrow 98, into the receiving channel 84 and aligned by the projections 94 seating within the corresponding receiving grooves or channels 94′. The selector coupling or connector 72 may be secured to the opening of the fluid reservoir 70 and the coupling or connector 76 may be secured to the terminal end of the flow selector 74. The flow selector 74 may be rotated about its longitudinal axis, as indicated by the rotational arrow 96, by the user or another individual to select one of the appropriate openings 88, 90, 92 of the flow selector 74 for alignment with the flow opening 86. When rotated within the receiving channel 84, the flow selector 74 may provide a tactile indication of when the projections 94 become seated within their corresponding grooves or channels 94′ which may also provide a tactile indication that the selected opening 88, 90, 92 of the flow selector 74 is correspondingly aligned with the flow opening 86. By rotating the flow selector 74 accordingly, the flow rate from the flow reservoir 70 may be adjusted depending upon the feeding needs of the infant. For instance, aligning the first opening 88 with the largest diameter to the flow opening 86 may allow for the largest flow rate from the fluid reservoir 70 which aligning the third opening 92 with the smallest diameter to the flow opening 86 may allow for the smallest flow rate from the fluid reservoir 70.

A flow selector 74 having any number of openings defined through the body and any number of projections 94 may be used with a corresponding selector coupling or connector 72 in any combination with any of the fluid reservoirs, fluid lines, nipple shields, etc. described herein in any number of variations depending upon the desired combination of features and are within the scope of this disclosure.

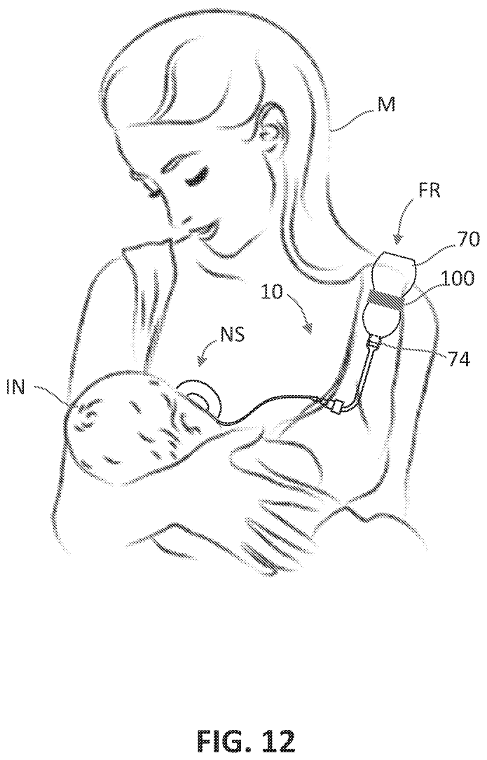

FIG. 12 shows an example of a nursing individual M breastfeeding an infant IN. The device is shown where the fluid reservoir assembly FR may be temporarily attached to the individual M such as via a securement strap 100 or other mechanism attaching the fluid reservoir 70 to the individual M such as their chest or a garment worn by the individual. The nipple shield assembly NS is shown with the nipple shield 12 adhered to the breast and selectively oriented relative to the positioning of the infant IN. Additionally, the fluid reservoir 70 is shown positioned at a location above the infant IN along the individual M although the fluid reservoir 70 may be located on a stand or holder separate from the individual M.

Furthermore, with the fluid reservoir assembly FR secured and configured to function without manual actuation and the nipple shield assembly NS secured along the breast, the device may be fully operational in a handsfree manner where the individual M is able to maintain both hands free for handling the infant IN.

Each and every feature described may be used in any number of combinations as practicable. For instance, any variation of the fluid reservoir assembly FR described may be used with any variation of the nipple shield assembly NS including variations on the crown or cavity 14, nipple shield 12, orientation indicator 66, opening configurations, fluid reservoir assemblies, etc.

While illustrative examples are described above, it will be apparent to one skilled in the art that various changes and modifications may be made therein. Moreover, various apparatus or procedures described above are also intended to be utilized in combination with one another, as practicable. The appended claims are intended to cover all such changes and modifications that fall within the true spirit and scope of the invention.

What is claimed is:

1. A breastfeeding aid device, comprising:
a reservoir having a first opening;
a flow selector positioned within the first opening, wherein the flow selector is configured to provide a predetermined flow rate from the reservoir;
at least one fluid lumen fluidly coupled to the distal opening of the reservoir;
a nipple shield configured to be positioned over a nipple of a subject such that the nipple is sealingly contained within a cavity defined by the nipple shield, the cavity having a channel fluidly coupled to the distal opening of the reservoir via the at least one fluid lumen and the cavity having at least one additional channel defined through a wall of the cavity, and
wherein a suction force applied upon an exterior of the cavity draws a fluid from within the reservoir and through the channel via the at least one fluid lumen and simultaneously creates a negative pressure within the cavity to stimulate the nipple of the subject.

2. The device of claim 1 further comprising a valve positioned within a second opening of the reservoir, wherein the valve is configured for a unidirectional flow of air into the reservoir.

3. The device of claim 2 wherein the valve comprises a unidirectional valve.

4. The device of claim 1 wherein the reservoir comprises a syringe body.

5. The device of claim 1 wherein the reservoir comprises a flexible pouch.

6. The device of claim 1 wherein the reservoir defines a plurality of gradations for indicating fluid volume.

7. The device of claim 1 further comprising an adjustable clamping mechanism positioned along the at least one fluid lumen for metering a flow of fluid through the at least one fluid lumen.

8. The device of claim 1 further comprising an additional fluid lumen fluidly coupled to the at least one fluid lumen.

9. The device of claim 1 wherein the cavity of the nipple shield is shaped into a nipple configuration.

10. The device of claim 1 wherein the nipple shield is configured to collapse its shape into a relatively wider configuration when compressed externally.

11. The device of claim 10 wherein the nipple shield has a first thickness along a first portion and a second thickness along a second portion adjacent to the first portion and where the second thickness is less than the first thickness.

12. The device of claim 1 wherein the nipple shield defines an indicator corresponding to a location of the channel fluidly coupled to the distal opening of the reservoir.

13. The device of claim 1 wherein the cavity defines a plurality of additional channels defined through the wall of the cavity.

14. The device of claim 1 further comprising a circumferential seal configured for placement around the cavity for contacting a skin of the patient.

15. The device of claim 1 further comprising an adhesive or sealant layer upon the nipple shield around the cavity.

16. The device of claim 1 wherein the at least one fluid lumen is configured to collapse upon application of the negative pressure applied within the cavity.

17. The device of claim 1 wherein the flow selector defines one or more openings through a body of the flow selector and where each of the one or more openings have a diameter different from one another.

18. The device of claim 17 further comprising a selector coupling or connector defining a flow opening and configured to receive the flow selector in an alignment corresponding to the one or more openings.

19. A method of aiding breastfeeding, comprising:
securing a nipple shield over a nipple of a subject such that the nipple is sealingly contained within a cavity defined by the nipple shield;
receiving a suction force imparted upon an exterior of the cavity such that a fluid contained within a reservoir is drawn via a negative pressure through at least one fluid lumen fluidly coupled to the reservoir and through a channel defined within a wall of the cavity while the fluid is metered via a unidirectional valve positioned within the reservoir; and
further receiving the suction force within the cavity such that the nipple of the subject is simultaneously stimulated.

20. The method of claim 19 further comprising adjusting a flow selector in fluid communication with the reservoir to provide a predetermined flow rate from the reservoir.

21. The method of claim 19 further comprising adjusting a clamping mechanism positioned along the at least one fluid lumen such that a flow of fluid through the at least one fluid lumen is metered.

22. The method of claim 19 wherein securing the nipple shield further comprises orienting the nipple shield to position the channel defined within the wall of the cavity prior to receiving the suction force.

23. The method of claim 19 wherein securing the nipple shield over the nipple comprises adhering the nipple shield to a skin surface around the nipple.

24. The method of claim 19 wherein imparting the suction force comprises collapsing the at least one fluid lumen when the negative pressure is applied within the cavity.

25. The method of claim 19 wherein the cavity of the nipple shield is shaped into a nipple configuration.

26. The method of claim 19 wherein the cavity defines a plurality of additional channels defined through the wall of the cavity.
